(19) United States
(12) Patent Application Publication
(10) Pub. No.: US 2024/0315680 A1
(43) Pub. Date: Sep. 26, 2024

(54) WEARABLE FOOT CONTROLLER FOR SURGICAL EQUIPMENT AND RELATED METHODS

(71) Applicant: ORASI LLC, LONGWOOD, FL (US)

(72) Inventor: Oliver Filutowski, Tampa, FL (US)

(21) Appl. No.: 18/733,192

(22) Filed: Jun. 4, 2024

Related U.S. Application Data
(63) Continuation-in-part of application No. 18/160,470, filed on Jan. 27, 2023, now Pat. No. 12,023,206.
(60) Provisional application No. 63/268,323, filed on Feb. 22, 2022.

(51) Int. Cl. A61B 17/00; A61B 90/20

(52) U.S. Cl. CPC A61B 17/00; A61B 90/20; A61B 2017/00973

(57) ABSTRACT

A wearable foot controller for a surgical instrument may include a body configured to be worn on a foot and including a heel portion and a ball portion on an opposite end of the body from the heel portion, a heel pressure sensor, a ball pressure sensor, a heel movement sensor, a ball movement sensor, and a processor. The processor may be configured to generate control data for the surgical instrument based upon the ball movement sensor responsive to an activation state the heel pressure sensor, and based upon the heel movement sensor responsive to an activation state of the ball pressure sensor. The processor may also be configured to communicate the control data from the processor to the surgical instrument via a communications link.

### CROSS-REFERENCE TO RELATED APPLICATIONS

This application is a continuation-in-part of U.S. application Ser. No. 18/160,470 filed Jan. 27, 2023 which claims the benefit of U.S. application Ser. no. 63/268, 323 filed Feb. 22, 2022, which are hereby incorporated herein in their entireties by reference.

### TECHNICAL FIELD

The present disclosure generally relates to surgical equipment, and more particularly to controllers for surgical equipment such as microscopes and related methods.

### BACKGROUND

In certain surgical applications, viewing aids are important to help surgeons see fine details and perform precise maneuvers. One such application is in eye surgeries, where a microscope is used to give the surgeon the ability to see acutely and perform delicate steps in the surgical process. Typically, the microscope is controlled by a large, mechanical foot pedal, allowing the surgeon to use his or her hands to operate surgical devices or tools. However, such foot pedals may be somewhat difficult to use. Moreover, they are typically heavy and difficult to move while cleaning the surgery room and reposition for a subsequent surgery.

European Pat. Pub. EP3744286A1 is directed to a microscope system that includes a surgical microscope, an operating device configured to be operated by a user, and a processor configured to receive a command signal from the operating device in response to the user operation and to control the surgical microscope based on the command signal. The operating device comprises a sensor unit configured to detect the user operation and to generate the command signal based on the detected user operation, and the sensor unit is further configured to be worn by the user.

Despite the existence of such systems, further developments in surgical equipment controllers may be desirable in certain applications.

### SUMMARY

A wearable foot controller for a surgical instrument may include a body configured to be worn on a foot and including a heel portion and a ball portion on an opposite end of the body from the heel portion, a heel pressure sensor coupled to the heel portion of the body, a ball pressure sensor coupled to the ball portion of the body, a heel movement sensor coupled to the heel portion of the body, a ball movement sensor coupled to the ball portion of the body, and a processor coupled to the body. The processor may be configured to generate control data for the surgical instrument based upon the ball movement sensor responsive to an activation state of the heel pressure sensor, and based upon the heel movement sensor responsive to an activation state of the ball pressure sensor. The processor may also be configured to communicate the control data from the processor to the surgical instrument via a communications link.

In an example embodiment, the processor may be further configured to activate the ball movement sensor responsive to activation of the heel pressure sensor, and activate the heel movement sensor responsive to activation of the ball pressure sensor. In accordance with another example embodiment, the processor may be configured to activate the ball movement sensor responsive to deactivation of the ball pressure sensor, and activate the heel movement sensor responsive to deactivation of the heel pressure sensor.

In one example implementation, the surgical instrument may be a microscope, and the control data may relate to at least one of pan, tilt, zoom, illumination and focus of the microscope. In some embodiments, the control data may relate to image recording using the microscope.

In some embodiments, the processor may be operable in a learning mode to associate at least one movement detected from at least one of the heel and ball movement sensors with a given control command. In some configurations, the processor may be further configured to change a weight associated with the control data based upon an adjustable sensitivity ratio. In an example implementation, the processor may be operable in an instructor mode in which the determined control commands override control commands from another surgical instrument controller.

In some configurations, the processor may be further configured to detect a series of movements from at least one of the heel and ball movement sensors, and switch between active and inactive states responsive to the series of movements. By way of example, the communications link may comprise a wireless communications link. In some embodiments, the wearable foot controller may further include a haptic feedback generator carried by the body and coupled to the processor, and the processor may be configured to operate the haptic feedback generator responsive to at least one of the heel pressure sensor, the ball pressure sensor, the heel movement sensor, and the ball movement sensor.

A related method for controlling a surgical instrument using a wearable foot controller, such as the one described briefly above, is also provided. The method may include generating control data for the surgical instrument based upon the ball movement sensor responsive to an activation state of the heel pressure sensor, and based upon the heel movement sensor responsive to an activation state of the ball pressure sensor. The method may further include communicating the control data from the wearable foot controller to the surgical instrument via a communications link.

A related non-transitory computer-readable medium for a wearable foot controller, such as the one described briefly above, is also provided. The non-transitory medium may have computer-executable instructions for causing the processor of the wearable foot controller to perform steps including generating control data for the surgical instrument based upon the ball movement sensor responsive to an activation state of the heel pressure sensor, and based upon the heel movement sensor responsive to an activation state of the ball pressure sensor. The steps may further include communicating the control data to the surgical instrument via a communications link.

### BRIEF DESCRIPTION OF THE DRAWINGS

FIG. 1 is a schematic block diagram of a wearable foot controller for surgical instruments in accordance with an example embodiment.

FIG. 2 is a perspective view of the bottom of an example implementation of the wearable foot controller of FIG. 1.

FIG. 3 is a perspective front view of the wearable foot controller of FIG. 2.

FIG. 4 is a bottom view of the wearable foot controller of FIG. 2.

FIGS. 5-7 are flow diagrams illustrating method steps which may be performed by the wearable foot controller of FIG. 1.

FIG. 8 is a schematic block diagram of another implementation of the wearable foot controller of FIG. 1.

FIG. 9 is a flow diagram illustrating haptic feedback operations which may be performed by the wearable foot controller of FIG. 8 in some embodiments.

### DETAILED DESCRIPTION

The present description is made with reference to various example embodiments. However, many different embodiments may be used, and thus, the description should not be construed as limited to the embodiments set forth herein. Rather, these embodiments are provided so that this disclosure will be thorough and complete. Like numbers refer to like elements or steps throughout.

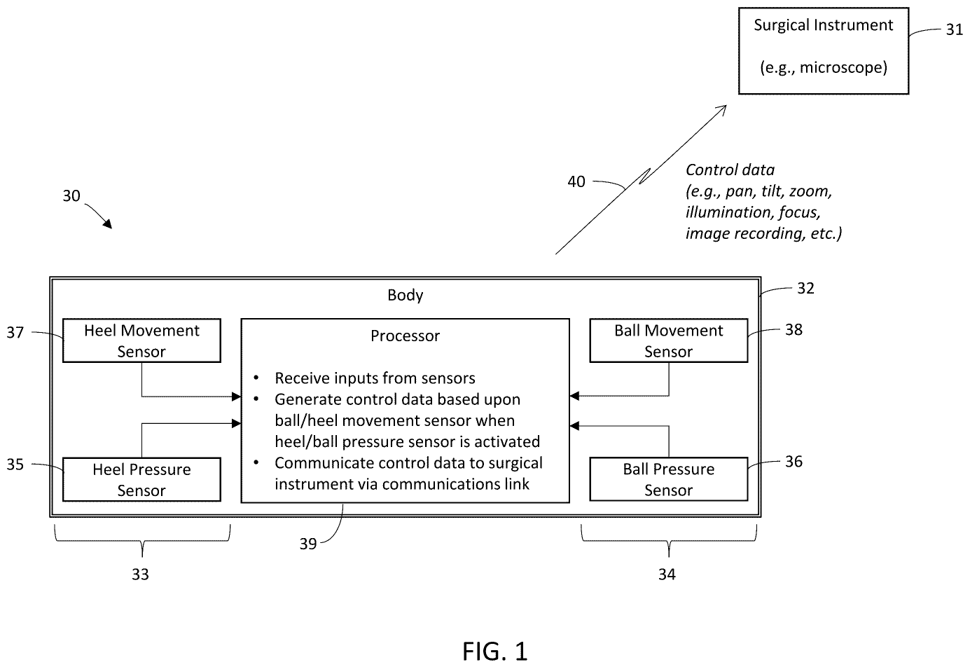

Referring initially to FIG. 1, a wearable foot controller or device 30 for controlling a medical instrument 31, such as a microscope for surgical eye procedures (e.g., laser eye surgeries, etc.), is first described. The wearable foot controller 30 illustratively includes a body 32 configured to be worn on a foot of a user and including a heel portion 33 and a ball portion 34 on an opposite end of the body 32 from the heel portion. A heel pressure sensor 35 is coupled to the heel portion 33 of the body 32, a ball pressure sensor 36 is coupled to the ball portion 34 of the body, a heel movement sensor 37 is coupled to the heel portion of the body, and a ball movement sensor 38 is coupled to the ball portion of the body. The wearable foot controller 30 further illustratively includes a processor 39 coupled to the body 32. The processor 39 may be implemented using suitable hardware (e.g., microprocessor, etc.) and non-transitory computer-readable medium having computer-implemented instructions for causing the hardware to perform the various operations discussed herein. The sensors 35-38 and processor 39 may be wholly or partially embedded within the body 32 or attached to a surface thereof in different embodiments.

More particularly, the processor 39 is configured to detect activation of the heel pressure sensor 35 and generate control data for the surgical instrument 31 based upon the ball movement sensor 38 when the heel pressure sensor is activated. Furthermore, the processor 39 also detects activation of the ball pressure sensor 36 and generates control data for the surgical instrument 31 based upon the heel movement sensor 37 when the ball pressure sensor is activated. Furthermore, the processor 39 is also configured to communicate the control data to the surgical instrument 31 via a communications link 40. In an example implementation, the communications link may be wireless (e.g., Wi-Fi, ultrawide band (UWB), Bluetooth®, etc.), although a wired connection to the surgical instrument 31 may be used in some embodiments.

In this regard, the processor 39 may further include (or have associated therewith) a transmitter or transceiver (e.g., Wi-Fi, UWB, Bluetooth®, etc.) to communicate measurements from one or more of the sensors 35-38 to the controller for the surgical instrument 31. Based upon this output, a controller at the surgical instrument 31 may control various operating parameters or functions. That is, the control data sent by the processor 39 may take the form of measurements, which the surgical instrument 31 controller interprets as different commands for functions to be performed by the surgical instrument. In other embodiments, the processor 39 may interpret the measurements from the sensors and supply the associated commands to the controller of the surgical instrument 31. In the case of a surgical microscope, these functions may include both the movement and the focus of the microscope, pan, tilt, zoom, illumination and in some cases image (video) recording using the microscope. For wireless implementations, a battery (ies) may also be carried by the body 32 for powering the processor 39 and sensor 35-38, and for wired implementations power may be supplied by batteries and/or the wired connection to the surgical instrument 31. Other example surgical instruments which may be controlled by the wearable foot controller 30 include phacoemulsification devices, cautery devices, vitrectomy devices, and indirect laser ophthalmoscopy (these devices are handheld and used by the surgeon, but each has a foot controller for utilization of these technologies). Being able to further adjust their settings and augment their power configurations with a foot controller (rather than requiring a human technician to press the buttons) may be advantageous in various implementations.

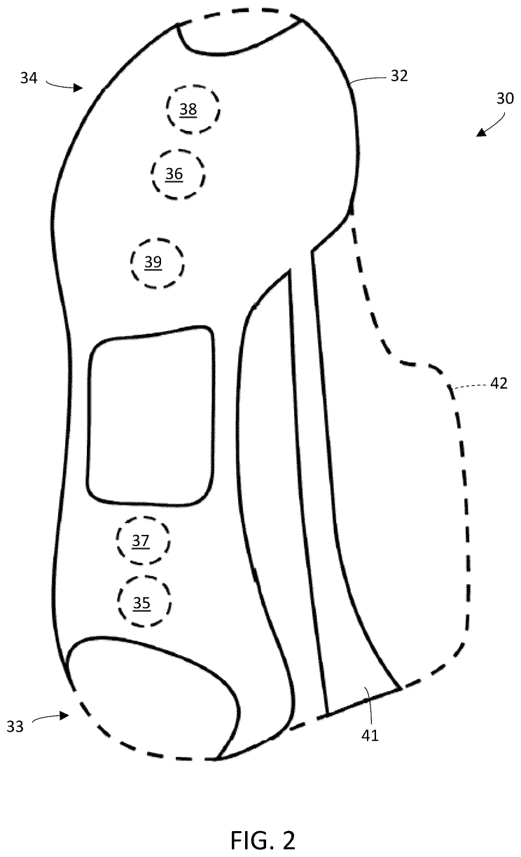

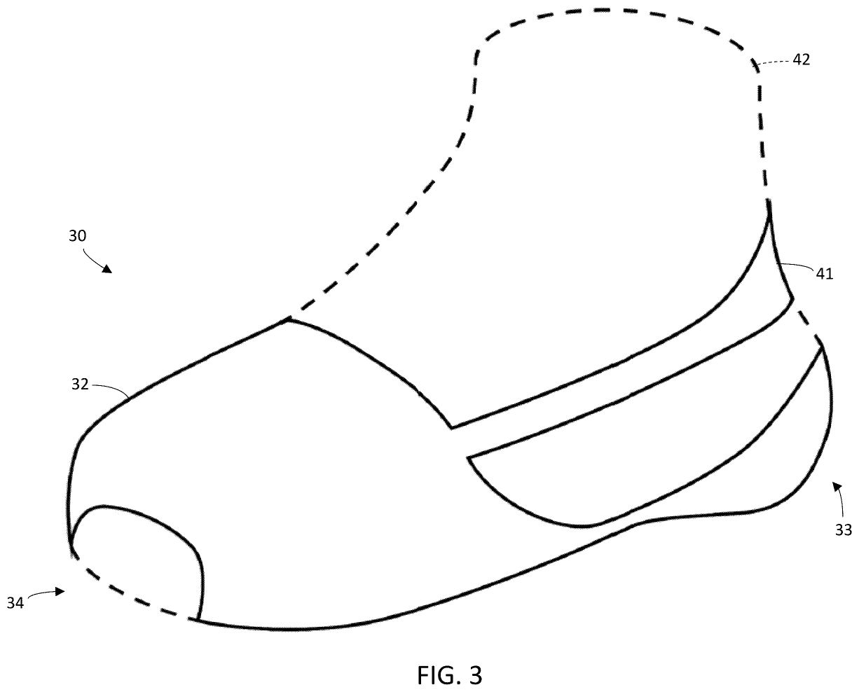

Referring additionally to FIGS. 2-4, in an example implementation the body 32 takes the form factor of a crampon with a heel strap 41 to fit over a shoe 42. In other embodiments, the body 32 may take the form of a sleeve/sock that fits on the user's foot, or it may have a form factor that resembles a sandal that can similarly fit on the foot or over the user's shoe. In other words, the body 32 may generally take the form of a relatively lightweight surgical foot controller that may be attached to or worn on the user's foot or over a shoe.

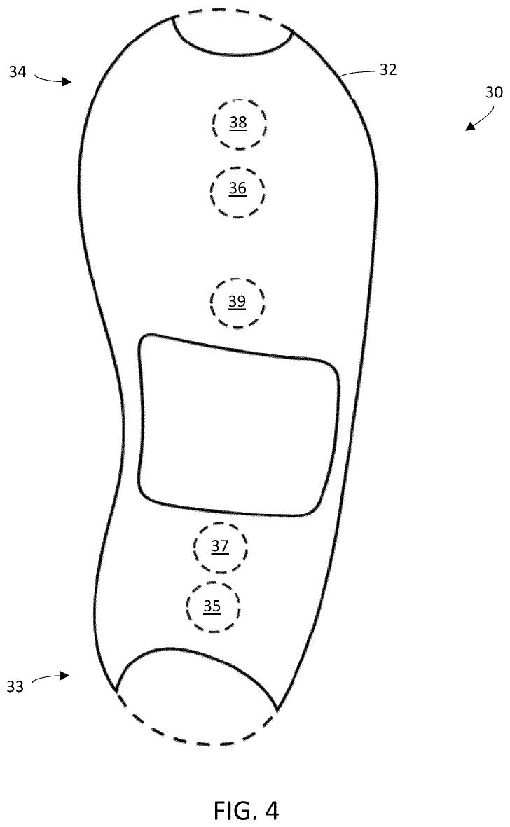

By way of example, the movement or motion sensors 37, 38 may include one or more of accelerometers, gyroscopes, etc., and the pressure sensors 35, 36 may take the form of pressure sensitive switches/touch pads. For example, touch pad pressure sensors 35, 36 may be located on the bottom of the body 32 (see FIGS. 2 and 4) to detect when a portion of the body is pressed against the floor. In the illustrated example where respective movement sensors 37, 38 and touch pad pressure sensors 35, 36 are associated with the heel and the ball/toe of the user's foot, detection of whether the user has the heel and/or ball of his or her foot on the ground, and whether the heel and/or ball is being moved, can be determined to provide enhanced control capabilities, as will be discussed further below.

By way of example, the following is a list of how foot movements detected by the motion sensors 37, 38 and touch pad pressure sensors 35, 36 may correlate with corresponding adjustments of a microscope:
Slide foot forward/backward/right/left->move scope forward/backward/right/left like joystick
Lift heel and tilt on ball of foot left and right->focus in and out
Lift ball of foot and tilt on heel of foot left and right->zoom in and out
Sweep ball of foot left and right (while keeping heel fixed)->turn brightness up/down
Double tap whole foot->on/off
Double tap ball of foot->re-center microscope
Double tap heel of foot->scroll through lighting settings
Movement for moving in and out of quiescent and active modes (e.g., a “double tap whole foot” while “triple tap whole foot” would be on/off)

In some embodiments, activation of the pressure sensor 36 at the ball of the foot may activate the corresponding sensor (s) 37 (e.g., accelerometer/gyroscopes) on the heel and suppresses (or deactivates) the sensor (s) 38 at the toe, and vice-versa. That is, to avoid potential interference or contradictory input from the heel and toe sensors, the processor 39 may utilize input from one of the touch/pressure sensors 35, 36 as a trigger to block or ignore input from sensors 37, 38 on the same end of the wearable foot controller 30.

In some example implementations, such as in training institutions, a plurality of wearable foot controllers 30 may be used with the surgical instrument 31. For example, an “attending physician” wearable foot controller 30 may be used, in addition to a wearable foot controller for residents, for training of the residents. In this way, a supervisor may take over control of the surgical instrument 31 (e.g., microscope) during a training session, such as to reorient the trainee's microscope if the trainee gets into trouble. For such an application, the system would allow one of the wearable foot controllers 30 to be designated as the trainee device, and the other as the instructor device such that it is given priority over the trainee device when it provides input to the microscope. Both wearable foot controllers 30 would include their own respective sensors 35-38 and processor 39, as discussed further above, and a controller at the microscope will interface with both and assign priority to the movements from the instructor wearable when appropriate. In some embodiments, the surgical instrument 31 controller may be integrated in the surgical instrument, and in other embodiments it may be a separate controller that connects to an input (s) of the surgical instrument and is configured to interface with the wearable foot controller 30, as noted above.

In accordance with another example aspect, a foot gesture may be used to change the wearable foot controller 30 from a quiescent state (in which foot movements do not result in surgical instrument 31 responses) to an active state (in which foot movements cause corresponding surgical instrument responses). For example, the foot gesture may be to briefly lift one's foot and place it back down. Once activated, the wearable foot controller 30 may remain in an active state until the same gesture is repeated or a separate gesture is performed, returning it to the quiescent state. This would enable the user to choose when the surgical instrument 31 would respond to foot movements and when it would not.

Use of the above-described wearable foot controller 30 to control operation of the surgical instrument 31 may provide several benefits. For example, these may include:
Improved dexterity;
Proper fit for the user (e.g., someone with a small foot using a large traditional foot pedal can struggle compared to someone with a large foot);
More space beneath the head of the patient's bed where the legs of the surgeon (s) are positioned for operating (this area is congested with multiple surgeons and their limbs and difficult to access for taller surgeons whose knees strike the bed due to the large height of traditional foot pedals);
Enhanced portability;
Less likely to get in the way while cleaning and preparing the operating room between patients; and
The ability to detect and assign new movement combinations for enhanced control capabilities.

Method aspects associated with controlling the

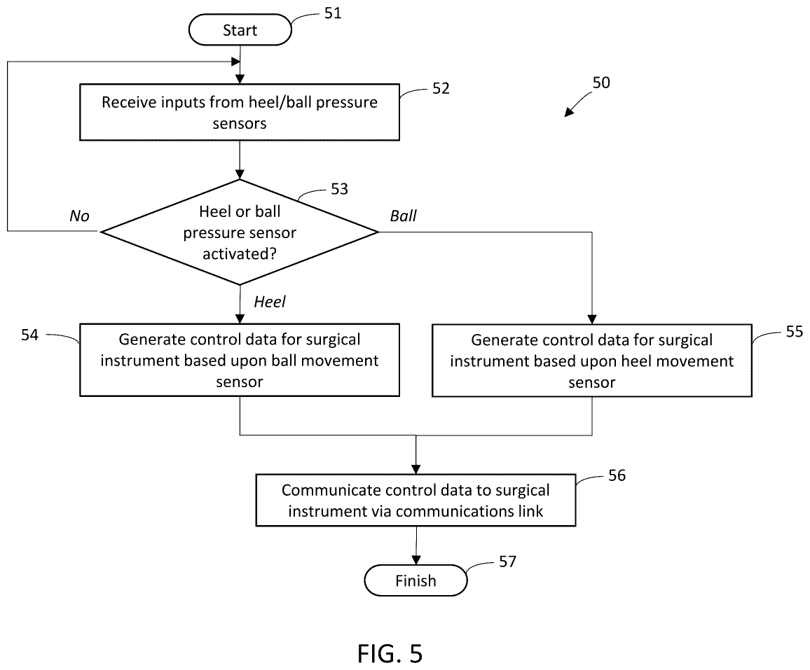

surgical instrument 31 using the wearable foot controller 30 are now further described with reference to the flow diagrams 50, 60, 70 of FIGS. 5-7, respectively. Beginning at Block 51, the processor 39 receives input from the heel and ball pressure sensors 35, 36 (Block 52). Upon detection of activation of the heel pressure sensor 35 (Block 53), the processor 39 generates control data for the surgical instrument 31 based upon the ball movement sensor 38, at Block 54. For example, left/right movement detected by the ball movement sensor 38 may result in pan left/right control commands being sent to the surgical instrument 31 (or left/right measurements in embodiments where the surgical instrument 31 interprets the measurements and generates the pan commands). Similarly, when the processor 39 detects activation of the ball pressure sensor 36 (Block 53), it generates control data for the surgical instrument 31 based upon the heel movement sensor 37, at Block 55. For example, left/right movement detected by the heel movement sensor 37 may result in “focus out” and “focus in” control commands being sent to the surgical instrument 31 (or left/right measurements in embodiments where the surgical instrument 31 interprets the measurements and generates the focus commands). The processor 39 communicates the control data signals to the surgical instrument 31 via the communications link 40, as noted above, which may be done in real or near-real time, at Block 56. The method of FIG. 5 illustratively concludes at Block 57.

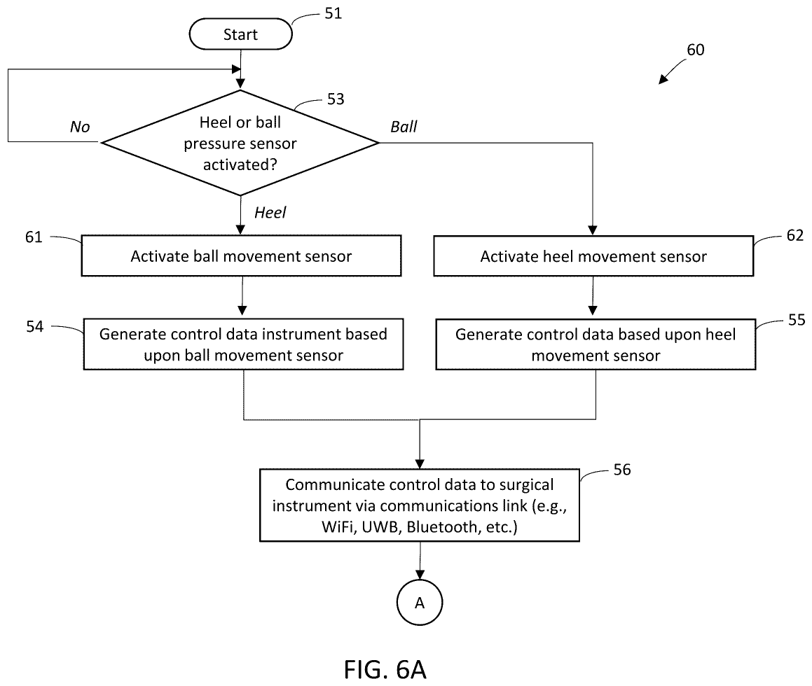

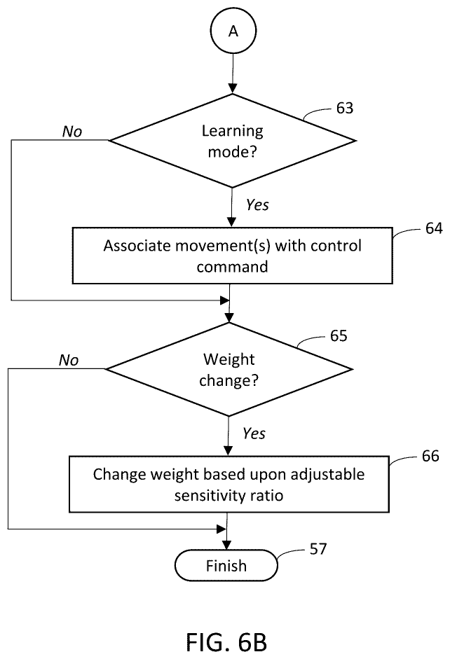

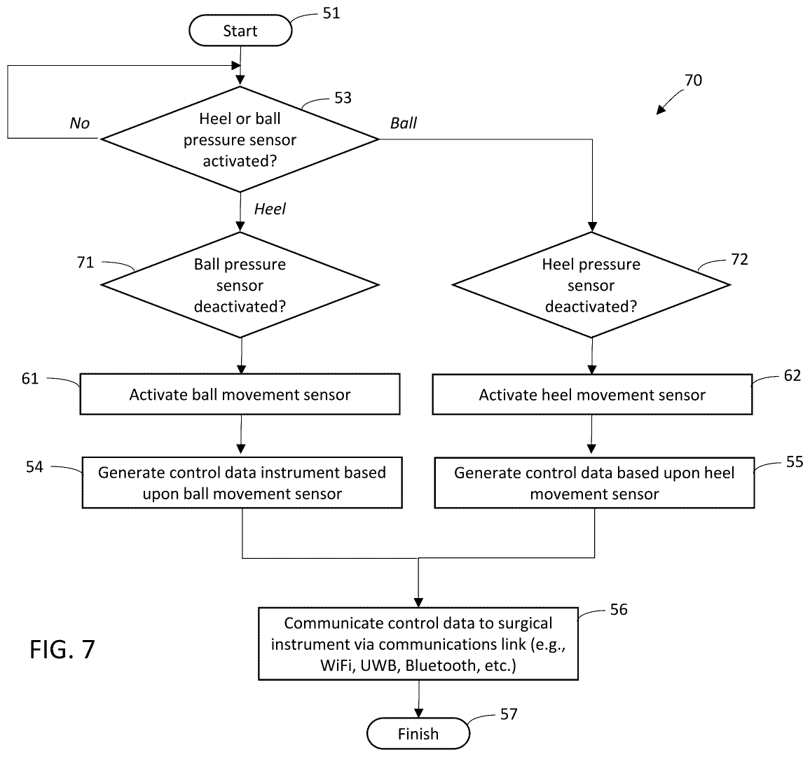

In the embodiment illustrated in FIGS. 6A and 6B, the processor 39 may further activate the ball movement sensor 38 responsive to activation of the heel pressure sensor 35 (Block 61), and activate the heel movement sensor 37 responsive to activation of the ball pressure sensor 36 (Block 62). This feature may be helpful for battery power savings, for example. Similarly, the heel and ball movement sensors 37, 38 may instead be activated responsive to deactivation of the heel and ball pressure sensors 35, 36, respectively (Blocks 71 and 72 of FIG. 7), which again may be beneficial for battery power savings, for example.

Furthermore, the processor 39 may be operable in a learning mode to associate one or more movements detected from the heel and/or ball movement sensors with a given control command, at Blocks 63-64. This may allow the user to define “macros” to perform a series of steps with less movements or at the same time, such as to zoom in a focus the microscope together. In some configurations, the processor 39 may be further configured to change a weight associated with the control data based upon an adjustable sensitivity ratio, at Blocks 65-66. More particularly, this may allow the user to adjust the granularity or sensitivity of movement he or she wishes to use to generate function commands for the surgical instrument 31 to personalize the user experience.

In some embodiments, the pressure sensors/touch pads 35, 36 alone (i.e., without a corresponding movement detected by the movement sensors 37, 38) may result in an output for controlling the medical instrument 31. More particularly, in some implementations certain foot positions/gestures may offer more reliable or consistent outputs when based upon the pressure sensors 35 or 36 as opposed to movement. For example, a “double tap” or “lift heel and tilt left and right on ball of foot” might be appropriate for pressure measurements alone versus gestures that are more appropriate for movement detection, like sliding a foot forward/backward/etc.

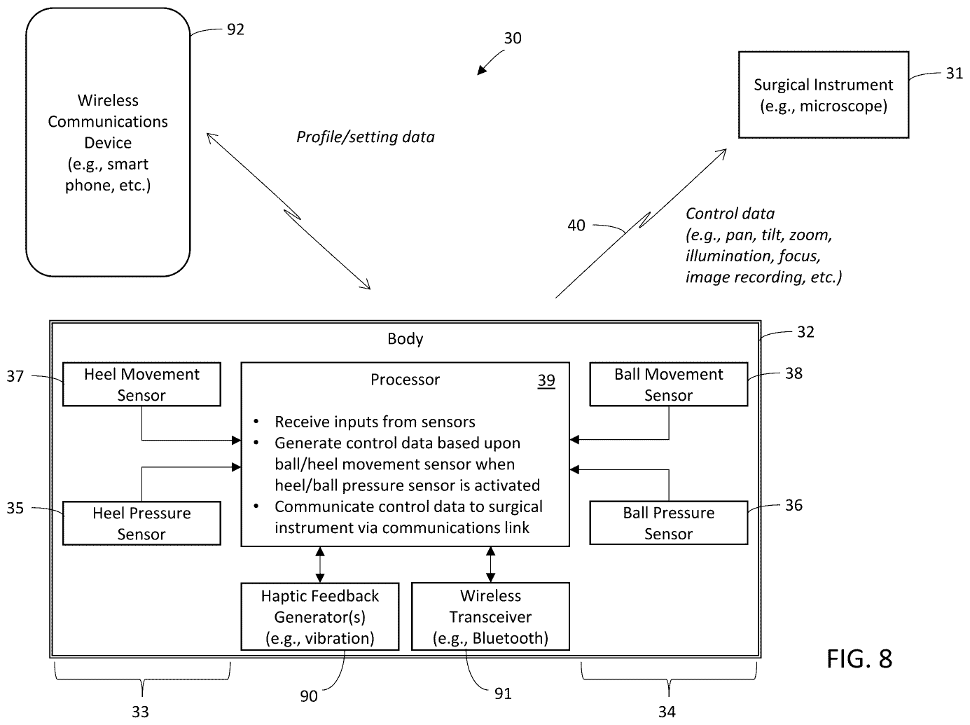

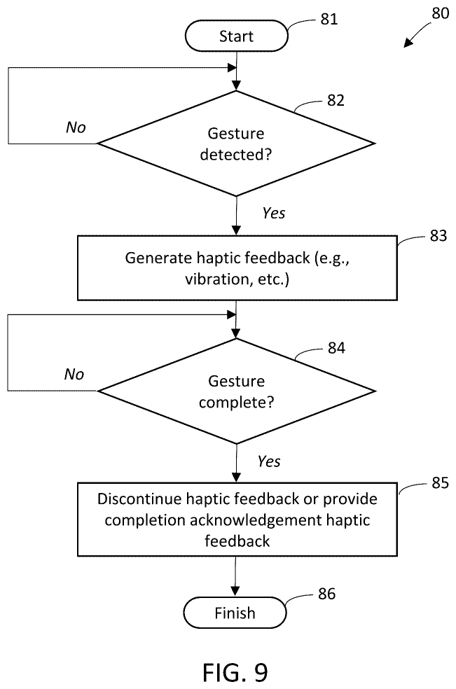

In accordance with another example embodiment now described with reference to FIG. 8 and the flow diagram 80 of FIG. 9, the wearable foot controller 30 may further incorporate haptic feedback, such as a rumble or vibration. Beginning at Block 81, when a gesture such as sliding is detected (e.g., sliding the foot forwards or backwards) by the processor 39 via the sensors 35-38 (Block 82), as discussed further above, a vibration may be provided (Block 83) via a haptic feedback generator 90 coupled to the processor 39 while this gesture is being performed, such as a continuous vibration or pulsed vibrations, for example. As soon as the foot stops moving and the gesture is complete (Block 84), the vibration would also cease (Block 85). Alternatively, the vibration may only be provided at the initiation (Block 83) and completion (Block 85) of a foot gesture, and not continue throughout the movement.

For other gestures such as tapping movements, a single vibration (or single group of vibrations, such as a “double tap”, etc.) may be used to acknowledge that the gesture command was received or executed, and accordingly that the microscope 31 is responding (e.g., double tapping the foot to turn on the microscope, double tapping the ball of the foot to recenter the microscope, etc.). By way of example, one or more vibration motors (e.g., linear actuators) may be carried by the body 32 and coupled to the processor 39 to provide the above described vibrational feedback, as will be appreciated by those skilled in the art. In other embodiments, different forms of haptic feedback may be used, such as air pressure, resistance, or heat, as will be appreciated by those skilled in the art. In some embodiments, more than one type of haptic feedback may be used, or multiple haptic feedback generators 90 may be used to provide feedback to different portions of the foot (e.g., heel, toe, left/right side, etc.).

In accordance with another example, haptic feedback may be provided to indicate operational movements of the surgical instrument 31. For example, surgical microscopes have a particular range of movement, zoom, focus, etc. In some embodiments, haptic feedback may be provided when the end of the range of the movement, zoom, focus, etc. is reached. For example, if the operator zooms in to the zoom limit of the microscope 31, haptic feedback (e.g., one or more rapid vibratory pulses) may be provided to indicate that the zoom limit has been reached.

In some embodiments, a wireless (e.g., Bluetooth, Wi-Fi, etc.) transceiver 91 coupled to the processor 39 may be used not only for communications with the surgical instrument 31, but also for communicating with a wireless communications device 92 (e.g., smart phone, tablet, computer, etc.) for setting profile or preference information of the wearable foot controller 30 for one or more users. Such settings may include movement sensitivity (e.g., speed and/or magnitude required to trigger commands), gesture definitions for different surgical instrument 31 actions, macros (e.g., a defined movement or shortcut to trigger a series of commands), reset time period between input commands, etc.

In an example implementation, a surgeon may create a profile via an app running on the wireless communications device 92 that allows the surgeon's settings to be customized and saved to his/her profile. In some embodiments, the wireless communications device 92 may instead communicate with the microscope 31, and the processor 39 receives profile settings from the microscope via the wireless transceiver 91. In either case, the surgeon is able to access his/her profile by logging into the app. For a surgeon to be linked to the microscope 31, the application may be opened and active. If there are two surgeons in the room, one operating and one observing, the observing surgeon would not have the application active on his/her respective device 92. Alternatively, there may be a specific button a surgeon can press in the app that allows his/her setting to generate priority and the other surgeon to be “bumped out,” e.g., to avoid a situation where someone accidentally interferes. Additionally, the surgeon's profile may be linked to the microscope 31 (as well as other microscopes/surgical devices). Generally speaking, it may be desirable to allow profile changes when the wearable foot controller 30 is in a “quiescent” or inactive mode, but not when in an active mode (although in some embodiments profile changes may be permitted in an active mode, if desired).

Many modifications and other embodiments will come to the mind of one skilled in the art having the benefit of the teachings presented in the foregoing descriptions and the associated drawings. Therefore, it is understood that the disclosure is not to be limited to the specific embodiments disclosed, and that modifications and embodiments are intended to be included within the scope of the appended claims.

1. A wearable foot controller for a surgical instrument comprising:
a body configured to be worn on a foot and including a heel portion and a ball portion on an opposite end of the body from the heel portion;
a heel pressure sensor coupled to the heel portion of the body;
a ball pressure sensor coupled to the ball portion of the body;
a heel movement sensor coupled to the heel portion of the body;
a ball movement sensor coupled to the ball portion of the body; and
a processor coupled to the body and configured to
generate control data for the surgical instrument based upon
the ball movement sensor responsive to an activation state of the heel pressure sensor, and the heel movement sensor responsive to an activation state of the ball pressure sensor, and
communicate the control data to the surgical instrument via a communications link.

2. The wearable foot controller of claim 1 wherein the processor is further configured to activate the ball movement sensor responsive to activation of the heel pressure sensor, and activate the heel movement sensor responsive to activation of the ball pressure sensor.

3. The wearable foot controller of claim 1 wherein the processor is further configured to activate the ball movement sensor responsive to deactivation of the ball pressure sensor, and activate the heel movement sensor responsive to deactivation of the heel pressure sensor.

4. The wearable foot controller of claim 1 wherein the surgical instrument comprises a microscope; and wherein the control data relates to at least one of pan, tilt, zoom, illumination and focus of the microscope.

5. The wearable foot controller of claim 1 wherein the surgical instrument comprises a microscope; and wherein the control data relates to image recording using the microscope.

6. The wearable foot controller of claim 1 wherein the processor is operable in a learning mode to associate at least one movement detected from at least one of the heel and ball movement sensors with a given control command.

7. The wearable foot controller of claim 1 wherein the processor is further configured to change a weight associated with the control data based upon an adjustable sensitivity ratio.

8. The wearable foot controller of claim 1 wherein the processor is operable in an instructor mode in which the determined control commands override control commands from another surgical instrument controller.

9. The wearable foot controller of claim 1 wherein the processor is further configured to detect a series of movements from at least one of the heel and ball movement sensors, and switch between active and inactive states responsive to the series of movements.

10. The wearable foot controller of claim 1 further comprising a haptic feedback generator carried by the body and coupled to the processor; and wherein the processor is configured to operate the haptic feedback generator responsive to at least one of the heel pressure sensor, the ball pressure sensor, the heel movement sensor, and the ball movement sensor.

11. A method for controlling a surgical instrument using a wearable foot controller, the wearable foot controller comprising a body configured to be worn on a foot and including a heel portion and a ball portion on an opposite end of the body from the heel portion, a heel pressure sensor coupled to the heel portion of the body, a ball pressure sensor coupled to the ball portion of the body, a heel movement sensor coupled to the heel portion of the body, and a ball movement sensor coupled to the ball portion of the body, the method comprising:
generating control data for the surgical instrument based upon
the ball movement sensor responsive to an activation state of the heel pressure sensor, and the heel movement sensor responsive to an activation state of the ball pressure sensor; and
communicating the control data from the wearable foot controller to the surgical instrument via a communications link.

12. The method of claim 11 further comprising activating the ball movement sensor responsive to activation of the heel pressure sensor, and activating the heel movement sensor responsive to activation of the ball pressure sensor.

13. The method of claim 11 further comprising activating the ball movement sensor responsive to deactivation of the ball pressure sensor, and activating the heel movement sensor responsive to deactivation of the heel pressure sensor.

14. The method of claim 11 wherein the surgical instrument comprises a microscope; and wherein the control data relates to at least one of pan, tilt, zoom, illumination and focus of the microscope.

15. The method of claim 11 wherein the surgical instrument comprises a microscope; and wherein the control data relates to image recording using the microscope.

16. The method of claim 11 wherein the wearable foot controller further comprises a haptic feedback generator carried by the body; and further comprising operating the haptic feedback generator responsive to at least one of the heel pressure sensor, the ball pressure sensor, the heel movement sensor, and the ball movement sensor.

17. A non-transitory computer-readable medium for a wearable foot controller comprising a body configured to be worn on a foot and including a heel portion and a ball portion on an opposite end of the body from the heel portion, a heel pressure sensor coupled to the heel portion of the body, a ball pressure sensor coupled to the ball portion of the body, a heel movement sensor coupled to the heel portion of the body, and a ball movement sensor coupled to the ball portion of the body, and a processor, the non-transitory medium having computer-executable instructions for causing the processor to perform steps comprising:
generating control data for the surgical instrument based upon
the ball movement sensor responsive to an activation state of the heel pressure sensor, and the heel movement sensor responsive to an activation state of the ball pressure sensor; and
communicating the control data to the surgical instrument via a communications link.

18. The non-transitory computer-readable medium of claim 17 further having computer-executable steps for causing the processor to activate the ball movement sensor responsive to activation of the heel pressure sensor, and activate the heel movement sensor responsive to activation of the ball pressure sensor.

19. The non-transitory computer-readable medium of claim 17 further having computer-executable steps for causing the processor to activate the ball movement sensor responsive to deactivation of the ball pressure sensor, and activate the heel movement sensor responsive to deactivation of the heel pressure sensor.

20. The non-transitory computer-readable medium of claim 17 wherein the surgical instrument comprises a microscope; and wherein the control data relates to at least one of pan, tilt, zoom, illumination and focus of the microscope.

21. The non-transitory computer-readable medium of claim 17 wherein the surgical instrument comprises a microscope; and wherein the control data relates to image recording using the microscope.

22. The non-transitory computer-readable medium of claim 17 wherein the wearable foot controller further comprises a haptic feedback generator carried by the body; and further having computer-executable instructions for causing the processor to operate the haptic feedback generator responsive to at least one of the heel pressure sensor, the ball pressure sensor, the heel movement sensor, and the ball movement sensor.
